(19) United States
(12) Patent Application Publication
(10) Pub. No.: US 2009/0276865 A1
(43) Pub. Date: Nov. 5, 2009

(54) Transgenic Drosophila line having fluorescent proteins with different colors

(75) Inventors: Ann-Shyn Chiang, Hsin Chu (TW); Tsai-Feng Fu, Wufeng Shiang (TW)

Correspondence Address: BACON & THOMAS, PLLC, 625 SLATERS LANE, FOURTH FLOOR, ALEXANDRIA, VA, 22314-1176, US

(73) Assignee: National Tsing Hua University, Hsin Chu (TW)

(21) Appl. No.: 12/385,726

(22) Filed: Apr. 17, 2009

Related U.S. Application Data
(63) Continuation of application No. 11/598,690, filed on Nov. 14, 2006, now abandoned.

(51) Int. Cl. A01K 67/00

(52) U.S. Cl. 800/13

(57) ABSTRACT

The present invention discloses a transgenic Drosophila line with genes of first fluorescent proteins and genes of second fluorescent proteins, wherein the genes of first fluorescent proteins are utilized to identify a predetermined area of tissues or a predetermined number of cells in the transgenic Drosophila line with a first exciting light source, and the genes of second fluorescent proteins are utilized to identify specific cells or neurons within the predetermined area of tissues or among the predetermined number of cells by illumination with a second exciting light source.

### RELATED APPLICATION

The present application is a continuation application of pending U.S. application Ser. No. 11/598,690, entitled “Method for Labeling Specific Cells within Living Cells or Tissues,” filed on Nov. 14, 2006, which is incorporated herein by reference in its entirety.

### FIELD OF THE INVENTION

The present invention relates to a transgenic Drosophila line, and more specifically, to a transgenic Drosophila line having fluorescent proteins with different colors for applications on Drosophila brain imaging and subsequent neurological analysis.

### BACKGROUND OF THE INVENTION

Biotechnology has had made remarkable contributions to the development of medical science rapidly. Some new inventions are already widely applied in biochemical engineering, cell culture culturing and materials science. The recent accomplishment of mapping the human genome, along with the development of bioinformatics, nanotechnology and proteomics, will eventually help us comprehend the mechanisms of genetic processes and diseases, and let us identify differences among individual humans, and the reasons why a certain therapy fails to work for someone. The ultimate objective of these researches is always to resolve the problems associated with human health. However, a pending problem is how to observe effectively gene distributions with particular functions within tissues or organs.

Since 1980, transgenic technology has allowed animals and plants to be utilized as tools for the medicinal protein production. Transgenic animals are produced when vectors with target genes are inserted into fertilized eggs or embryonic stem cells via microinjection. The target gene can enter the cell nucleus, resulting in gene recombination, thereby inserting target genes into chromosomes or interrupting nearby genes within chromosomes. These mutations occur in all cells following embryonic development and cell duplication. Microinjection and nuclear transfer technology are frequently used in the field for generating transgenic animals. A transgenic strain can be identified after the filial generation is born.

The most direct approach to identifying the functions of a target gene is to clone or mutate the target gene in an animal and assess the consequences. Therefore, there is a need for an immediate and simple method for target detection. However, no effective tool exists for labeling specific cells within the tissues and organs.

The extensively utilized green fluorescence protein (GFP) is derived from the fluorescence protein of Aequorea Victoria (jellyfish). Mutant mammalian cells inserted with the GFP gene emit stable fluorescence following excitation (Ex=488 nm, Em=507 nm). Additionally, the genes of green fluorescence proteins may be forward or backward introduced into target genes via gene recombination, and fusion proteins with the target protein and fluorescence protein can then be translated. Conversely, according to Brand and Perrimon (1993) (Development 118:401), an upstream activation sequence (UAS) can be attached to a GFP gene, with a transactivator (such as GAL4) initiated by specific genetic mechanisms (such as a gene trap, enhancer trap, or be promoter driven) from specific gene(s) to trigger the production of GFP. Therefore, green fluorescence protein can be used as a marker for expression or variation of genes or proteins within cells or tissues.

In addition to the green fluorescence protein, the red fluorescence protein was derived from Discosoma Striata (DsRed) in 1990, and subsequently modified by BD Biosciences and Clontech. The genes for red fluorescence proteins can also be inserted into mammalian cells to achieve intense and stable fluorescence (Ex=558 nm, Em=583 nm). The DsRed proteins have been demonstrated to facilitate the transformation of the fusion proteins into nucleuses, whereas the modified red fluorescence proteins developed by Clontech in 2001 (DsRed2) do not.

Moreover, certain mutant proteins, such as yellow fluorescence protein (YFP) (Ex=513 nm, Em=527 nm) and cyanine fluorescence protein (CFP) (Ex=433 nm, Em=475 nm), can also be used to label biological targets as the green fluorescence proteins. Multiple labels can be observed with appropriate fluorescence filters.

In 2002, the photoactivatable fluorescence protein (Pa-GFP) was discovered. After intense irradiation with ultraviolet light, the Pa-GFP enhances fluorescence, emitted at 507 nm, by about 100-fold when excited by 488 nm light. These various fluorescence proteins are different tool for gene labeling in cells. However, these proteins have not yet been combined with living tissues effectively.

Genetic and behavioral analysis has been developed to assist in identifying gene functions. For example, numerous approaches have been developed for disease modeling, such as the production of germ-line transgenic animal models (e.g., transgenic mice and other animals with specific genetic characteristics). However, a principal obstacle in performing gene/disease analysis on transgenic mammals is the long life span of animals. That is, it takes at least a few years in the laboratory to trace diseases evolving from abnormal genes in one animal. One remedy to this problem is to employ some relevant systems in insects with short life spans (only days from birth to mature) as models. For instance, the brain of fruit flies (Drosophila melanogaster) has been used to investigate the pathogenesis of Alzheimer's disease. Similarly, related studies investigating early detection and treatment of numerous diseases may experience increased efficiency in the future when a good correlation among genes, cellular structures and diseases can be established in a fly model. The fruit fly has become a primary model system in brain research. Its brain (approximately 600×250×150 micrometers (μm)) consists of about 200,000 neurons. Given its relatively small brain, the fly shows a markedly complex repertoire of behaviors, e.g., orientation, courtship, learning and memory. Whole fly brains were dissected from heads, sliced and labeled fluorescently for examination. However, when using this methodology, as in all prior methods, individual neurons within the whole 3D neural circuitry in the fly brain cannot be selectively presented and analyzed. The proposed invention effectively overcomes this barrier.

The invention, which is a method for labeling specific cells within transgenic tissues or among cells, has the advantages of green fluorescence protein genes that are easily expressed and the short life cycle of Drosophila. The genes for green fluorescence proteins and their variations are inserted into the Drosophila embryo for future observations. Additionally, based on experimental results for current studies of gene expression and bioimaging, the present invention provides useful organisms used for scientific observations.

### SUMMARY

Herein, the present invention will explain certain embodiments in detail. However, it is appreciated that the present invention can extensively apply to other embodiments expect for these explicit descriptions. The scope of the present invention is not limited to the above-mentioned embodiments, and the preferred embodiment is illustrative of the present invention rather than limiting the present invention.

The present invention is to produce the Drosophila of dual mutations, and create a novel species that presents a unanimous-distributed genetically drivable DsRed (such as UAS-DsRed) on one hand and the genetically drivable Pa-GFP (such as UAS-Pa-GFP), for specific investigations, on the other hand, under the control of a transactivator (such as GAL4) triggered by the expression of specific gene(s) in the whole brain. All neurons and live tissues expressing DsRed can be observed under a microscope so that areas or cells with specific genes (thus containing the label of Pa-GFP) may be roughly identified. After the activation of the 2-photon laser light at about 820 nm (which does not damage the living cells), only the specific areas will emit the intensive fluorescence excited by 488 nm light, regions above, beneath or surrounding the area will not be affected. The pathways of the specific neurons or cellular networks can be tracked due to the diffusive labeling of the Pa-GFP within the cell. The present invention has many features as follows, (1) the images of target cells can be identified and chosen ahead of time, (2) only the target cells (none above or beneath or surround) are photoactivated in live tissues, and (3) the spatial resolution of the image reveals the real physiological fine structure within the living tissue.

One advantage of the present invention is to provide a method for tracking transgenic organisms of, including but not limited to, Drosophila, and providing the transgenic Drosophila line with target genes to facilitate the exploration for influences and variations induced by target genes.

Another advantage of the present invention is to provide a method for labeling specific cells within transgenic tissues or among cells, including but not limited to, Drosophila. The genes of green fluorescence protein (GFP) are inserted into Drosophila embryos as the label by transgenic technology.

Still another advantage of the present invention is to provide a method for studying transgenic Drosophila through exploring the intracellular protein dynamics, movements and pathway by tracking the visible green fluorescence protein (GFP) within the cell.

Additional advantage of the present invention is to provide a method for labeling specific cells in transgenic Drosophila. The proteins within the living cells or tissues are visible due to the combination of green fluorescence protein (GFP) and homologues fluorescence protein. The present invention also provides the information regarding the intracellular protein movements and protein-protein interactions.

One object of the present invention is to disclose a method for labeling specific cells in living brain of transgenic Drosophila.

Another object of the present invention is to disclose a method for tracking the development of a target neuron and other connections among other neurons in transgenic Drosophila. The present invention is also used for labeling the establishment of any neural circuit in transgenic Drosophila brain.

The present invention discloses a method for labeling specific cells within living tissues or among cells, comprising preparing vectors with the genes of phtoactivatable fluorescent proteins, followed by injecting the vectors with the genes of phtoactivatable fluorescent proteins into living cells or tissues, and irradiating predetermined areas by an activation light source, subsequently, the duration and intensity of the fluorescent are enhanced.

The present invention further comprises preparing vectors with genes of non-phtoactivatable fluorescent proteins in order to present the whole structures of the living cells or tissues, and from there to pick and label neurons carrying the phtoactivatable genes, on site at the scene under a microscope.

The present invention discloses a transgenic Drosophila line with the genes of phtoactivatable fluorescent proteins, wherein the Drosophila line is irradiated by an activation light source with two-photon facility, so that the duration and intensity of the fluorescent emission can be improved after excitation but only target cells at designated spatial locations are engaged.

### BRIEF DESCRIPTION OF THE DRAWINGS

The patent or application file contains at least one drawing executed in color. Copies of this patent or patent application publication with color drawings will be provided by the office upon request and payment of the necessary fee.

The foregoing aspects and many of the attendant advantages of this invention will become more readily appreciated as the same becomes better understood by reference to the following detailed description, when taken in conjunction with the accompanying drawings, wherein:

FIG. 1 illustrates a flow chart of a method for labeling the PA-GFP and DsRed transgenic Drosophila according to one embodiment of the present invention.

FIG. 2 illustrates a diagram of image-guided labeling of living neuron with PA-GFP and DsRed in one live fly brain according to one embodiment of the present invention.

### DETAILED DESCRIPTION OF THE PREFERRED EMBODIMENT

The drawings and the following descriptions present and describe the purpose of illustrating the preferred embodiments of the present invention only, and are not for the purpose of limiting the present invention. The present invention provides the information for intracellular protein expression and neuron networks, and a preferred embodiment for protein expressions induced in Drosophila embryos. Such an expressing system should be modular to facilitate expansion for multiple proteins with different functions.

Several phtoactivatable proteins are disclosed. For instance, phtoactivatable green fluorescent protein (PA-GFP) (Science (297):1873, 2002), kindling fluorescent protein 1 (KFP1) (Nat Biotechnol. (21):192, 2003) and Kaede (PNAS, 2002) have been developed in recent studies.

The phtoactivatable protein introduced facilitates precise photolabeling and tracking of the protein, thereby, providing complete information of the protein's dynamics. The phtoactivatable protein (PA-GFP) is preferred to be monomer that avoids aggregation under the circumstances of protein trafficking.

In one preferred embodiment, PA-GFP, the phtoactivatable protein is a variant of the Aequorea Victoria green fluorescent protein that, after intense irradiation with about 413-nm light, increases fluorescence by 100-fold and when excited by 488-nm light and remains stable for days under aerobic conditions. Based on these PA-GFP characteristics, the present invention is a novel tool for exploring intracellular protein dynamics by tracking photoactivated molecules that are the only visible GFPs in the cell.

The present invention discloses a method for labeling specific cells within living tissues or among cells, comprising steps for preparing vectors with the genes of phtoactivatable fluorescent proteins, followed by injection of the vectors with genes of phtoactivatable fluorescent proteins into living cells or tissues; predetermined areas are irradiating by an activation light source, such that the chemical reaction can be generated for dynamic observation of cells and tissues. Moreover, the present invention further comprises a step of preparing vectors with the genes of non-phtoactivatable fluorescent proteins.

The characteristics of fluorescent proteins, and specific fluorescent emissions after excitation with light at a specific wavelength, meet the requirements of these applications; preferably the fluorescent proteins are phtoactivatable green fluorescent proteins, including PA-GFP that can be activated by irradiation with ultraviolet (UV) light. The present invention can be applied for tracking the development of a target neural cell and the connections between other neural cells, or the formation of neural circuits.

The present invention discloses a method for studying transgenic Drosophila, which comprises preparing Drosophila embryos and vectors with phtoactivatable fluorescent protein genes, followed by injecting the vectors with genes of phtoactivatable fluorescent proteins into embryos via microinjection, hybridizing the transgenic Drosophila having PA-GFP with the transgenic Drosophila that has DsRed after incubating for predetermined periods, selecting transgenic Drosophila PA-GFP and DsRed and incubating the embryos for the predetermined periods, and irradiating the predetermined areas in cells or tissues with an activation light source. Subsequently, the dynamics of phtoactivatable fluorescent proteins can be observed out of the non-phtoactivatable fluorescent proteins within cells or tissues.

A preferred embodiment is as follows. Herein, Drosophila is used as an experimental target for to illustrate the preferred embodiments of the present invention only, and not for the purpose of limiting the same.

### Preparation of the Experimental Target

Embryos at very early stages (1 or 2) are selected as the system for microinjection. One advantage of the present invention is that Drosophila is easy to incubate, and it has preferred light transparency and high tolerance to the environment, and the Drosophila chromosomes are massive and suitable for mutation research.

The present invention discloses a method by which phtoactivatable fluorescence proteins and target genes are inserted into the Drosophila embryos, therefore provides the transgenic D. melanogaster lines, as follows.

### Fluorescent Protein: Phtoactivatable Green Fluorescent Protein (PA-GFP) and Non-Phtoactivatable Red Fluorescent Protein (DsRed)

The phtoactivatable fluorescent protein (PA-GFP) and non-phtoactivatable fluorescent protein are hereafter referred to as PA-GFP and DsRed.

In the present invention, Green fluorescent protein is extracted from the fluorescence protein of Aequorea Victoria (jellyfish). The protein has visible fluorescent and is a suitable for labeling living cells due to its fluorescence and non-toxicity to living cells. Thus, green fluorescent protein is very suitable for labeling in the in vivo studies, such as those examining on gene expression, protein localization, secretion pathways, cellular organelles and cytoskeleton. Furthermore, a series of fluorescence proteins discovered from GFP mutants are blue fluorescence protein (BFP), cyanine fluorescence protein (CFP), yellow fluorescence protein (YFP), and red fluorescence protein (DsRed), etc. The proteins with different fluorescences can be used in various investigations with labels for different living tissue cells.

Alternatively, PA-GFP-A206K, a PA-GFP mutant containing one of the three substitutions (A206K) was identified by Dr. R. Tsien (Science (296):913, 2002). Even at high concentrations, this mutation disrupts dimerization of the fluorescent protein. The PA-GFP-A206K mutant does not exhibit obvious fluorescent differences compared with the original fluorescent intensity, however, the fluorescence diffusion rate is better than the original diffusion rate, in our own generated transgenic lines. Therefore, the preferred embodiment of PA-GFP is described as follows.

Herein, the red fluorescence protein, DsRed, is known to persons skilled in the art, so that the descriptions are omitted.

The present invention discloses that the fluorescence proteins with target genes are inserted into the Drosophila embryos and the transgenic D. melanogaster lines, as follows.

### Preparation of the Gene Vector

To investigate and identify the neuronal circuits and trace the tracing target neurons of interest, this work generated transgenic D. melanogaster lines expressing PA-GFP, under the control of the upstream activating sequence (UAS), with the following GAL4 protein binds on (Brand and Perrimon, 1993, Development 118:401).

First, unique primers, a forward primer 5′-ATGGTGAGCAAGGGC (SEQ ID NO: 1) and a reverse primer 5′-TTACTTGTACAGCTC (SEQ ID NO: 2), are used to generate the full-coding region of the PA-GFP or PA-GFP mutant (PA-GFP-A206K) by using the pPA-GFP-N1 and pPA-GFP-A206K vectors-kindly provided by Dr. J. Lippincott-Schwartz—as templates for polymerase chain reaction (PCR).

According to the above-mentioned method, the cDNA fragments of the PA-GFP or PA-GFP-A206K mutants are amplified by PCR and cloned into pGEM-T-easy TA cloning vector (Promega), respectively. The target genes are cloned into the plasmids of E. Coli for gene expression. The plasmids are cloned into the E. Coli via transformation and the target genes are expressed in E. Coli. After incubation of the plasmid colonies, the culture media with plasmids are collected and reacted with the restriction enzyme, EcoR I. The fragments of PA-GFP or PA-GFP-A206K mutant fragments are extracted from the pGEMT-T-easy vector containing a single EcoRI cutting site for the release of the PA-GFP (or PA-GFP-A206K) genes, and, hence, the PA-GFP gene is cloned into the pP[UAST] vector.

Herein, the red fluorescence protein, DsRed, and the method for preparing the same are known to persons skilled in the art, thus, the descriptions are omitted (e.g., Hideaki Mizuno, Asako Sawano, Pharhad Eli, Hiroshi Hama, and Atsushi Miyawaki, Red Fluorescent Protein from Discosoma as a Fusion Tag and a Partner for Fluorescence Resonance Energy Transfer, Biochemistry; 2001; 40(8) pp 2502-2510).

Based on the above-mentioned method, D. melanogaster expressing PA-GFP is generated by injecting pP[UAST-PA-GFP] or pP[UAST-PA-GFP-A206K] combined with a p-helper into the Canton-S (CS10, 2U) strain.

### Microinjection

The capillaries with known outside diameter (O.D) and inside diameter (I.D) suitable for Drosophila embryos are manipulated using a puller and become the needle-like with sharp tips (according to capillary sizes, and the parameters, such as air press, heat temp and pull speed, etc., are determined). The needles with sharp tips are ground into needles with a slight opening at a suitable cutting angle for microinjection.

The Drosophila embryos are placed in the petri dishes or on glass slides, and the positions of the embryos are fixed in the sequence. Next, the embryos are covered with culture oil to maintain embryo morphology during microinjection. Subsequently, the fragments with target genes are injected into embryo tails of the embryos using the fine needle by microinjection.

Next, the embodiment of the present invention illustrates that process of injecting vectors of fluorescent protein genes into embryos for observation and labeling of living cells or tissues. In the embodiment of the present invention, the gene vectors can include two fluorescent proteins with different colors, and not limited to green and red fluorescent proteins. The above-mentioned descriptions are only used to descript the present invention, but not limited to the present invention.

### Incubation

After incubating embryos for a predetermined period, the embryos are moved into other incubation containers with food to continue the incubation for the research period. The predetermined periods in the embodiment include various incubation periods for the developments of Drosophila brain. In the embodiment, the incubation processes for the embryos include incubating with culture oil and then moving the embryos into a food container for feeding, however, the incubation processes are not limited to the above-mentioned descriptions. The embodiment can be modified and varied according to the experiment requirements under the scope and spirit of the present invention. The process for raising transgenic Drosophila carrying the DsRed gene is known to people familiar with the art.

### Hybridization

The transgenic flies with the PA-GFP gene, driven by the specific genes (such as C133), can be selected via the hybridization between the transgenic flies with PA-GFP that can be driven (such as with the upstream activation sequence (UAS)) and the transgenic flies with the transactivator (such as GAL4) accompanied with specific genes (such as C133). The transgenic flies that simultaneously contain drivable PA-GFP (such as UAS-GFP) and drivable DsRed genes (such as UAS-DsRed) are selected after hybridization between different transgenic flies with drivable PA-GFP and drivable DsRed, respectively. This method is well known to the one of ordinary skills in the art. The book, titled “Fly Pushing—The theory and practice of Drosophila genetics” by R. J. Greenspan, printed by the Cold Spring Harbor Laboratory Press (1997) is a good reference.

### Irradiation

In the embodiment, the phtoactivatable protein PA-GFP-A206K, is a preferred embodiment of the PA-GFP protein, and is a variant of the Aequorea Victoria green fluorescent protein (PA-GFP). Following an intensive irradiation with about 413-nm light (or at other wavelengths with equivalent effects, e.g. about 820 nm from a two-photon laser), PA-GFP increases its fluorescence by approximately 100-fold when excited by 488-nm light, and remains stable for days under aerobic conditions. Based on these characteristics of PA-GFP, the present invention offers a new tool for exploring intracellular protein dynamics and cell tracing by tracking photoactivated molecules that are the only visible GFPs in the cell. This is especially the case when the two-photon system is employed, only a few neurons in the whole fly brain tissue are displaying GFP molecules. Neurons above, beneath or surround are not engaged.

### Observation and Recording

The samples of present invention are coupled to a three-dimensional stereo image processing system with a stereo project system that present full colored three-dimensional stereo images. For a detailed description of the image presentation, please refer to the related applications of a pending U.S. application Ser. No. 11/169,890 and Taiwan application No. 094113324, titled “Bio-expression system and the method of the same,” which is incorporated herein as reference in its entirety.

A full-colored three-dimensional stereo neuron graphic can be seen and manipulated with the following facilities. In order to reveal the very fine extension of neurites, several related facilities are provided. A Zeiss LSM 510 confocal microscope is equipped with four laser light sources including an argon laser (emission at 364 nm), an argon-krypton laser (458, 488, or 514 nm), and two HeNe lasers (543 and 633 nm). The system allows for simultaneous detection of four fluorescence signals and a transmitted image. The Zeiss LSM 510 META confocal two-photon microscope system is equipped with four laser light sources, including an argon-krypton laser (458, 488, or 514 nm), two HeNe lasers (543 and 633 nm), with a Coherent Mira femtosecond T-Sapphire laser for nonlinear optical microscopy (2-photon) that is capable of 700-1000 nm single optics set tuning. The system is designed for in vivo observation of fluorescence signals in thick living tissues. The Zeiss LSM 510 META confocal microscope is equipped with three laser light sources including an argon-krypton laser (458, 488, or 514 nm), and two HeNe lasers (543 and 633 nm). The system has three photomultipliers and a META detector allowing simultaneous collection of full spectrum fluorescence signals. Although, the system does not have the transmitted light detector, it has an automated stage scanner for image montage and optical system for detecting with infrared (IR) light.

For the stereoscopic image presentation, a stereoscopic projecting system is coupled to the process system. The process system can access the database under the input instructions and send the image to a video card with multiple graphic outputs (such as NVIDIA Quadro4-980 or better level graphic outputs). The CPU in the process system 100 can be a 32-bit or 64-bit (or better) unit(s), with sufficient memory for image data processing. The image from the multiple outputs is individually fed into multiple projectors so that a front or back projection can be implemented for stereoscopic presentation and manipulation. The procedure can be controlled by (but is not limited to) commercially available software (such as AMIRA v.3.1) and hardware (such as a 3D mouse). Special glasses as known in the art should be provided for generating the virtual three-dimension image. As the above glasses are well known in the art, the description is omitted.

Furthermore, in the embodiment, the methods can be modified according to experimental requirements. The different fluorescent images can be obtained using different filters for different injected fluorescent proteins.

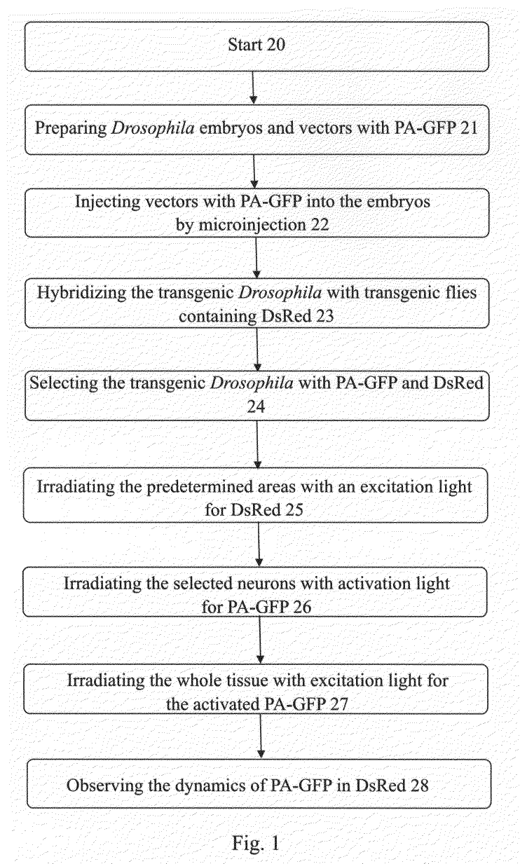

FIG. 1 illustrates a flow chart of a method for labeling the PA-GFP and DsRed transgenic Drosophila according to another embodiment of the present invention. The method 20 comprises the following steps. First, the Drosophila embryos and vectors with PA-GFP genes are prepared in step 21. The vectors with genes of PA-GFP fluorescent proteins are injected into the Drosophila embryos via microinjection in step 22. Next, the transgenic fly with PA-GFP hybridizes with the transgenic fly with DsRed in step 23. The transgenic fly with both PA-GFP and DsRed is selected and continuously incubated for the predetermined periods in step 24. Subsequently, some predetermined areas in cells or tissues are irradiated to illuminate the DsRed in step 25. Cells of interest are then chosen visually and irradiated with an UV light (or its equivalent) in step 26. The structure with activated PA-GFP is revealed by an irradiation for GFP in step 27. The dynamics of the fluorescent proteins PA-GFP in DsRed within the cells or tissues are observed in step 28.

In one embodiment, the whole structures of living cells or tissues can be identified by the non-phtoactivatable protein, DsRed, after excited by an excitation with a light source, and the wavelength of the excitation light source is approximately 558 nm.

In one embodiment, the dynamics of the fluorescent proteins include, but are not limited to, intracellular protein dynamics, velocities and interactions. The predetermined areas in cells and tissues include, but not limited to, neural cells or the connections between neural cells and neural circuit systems.

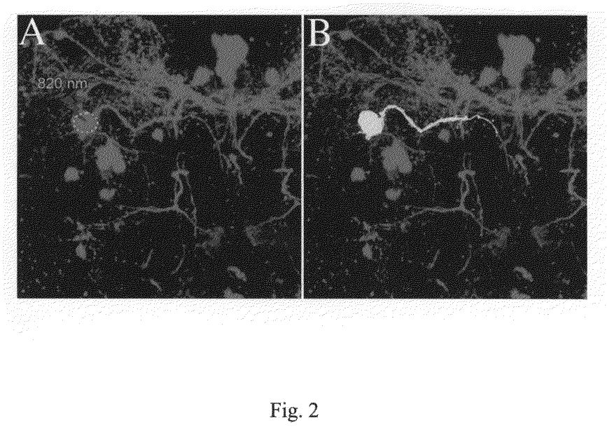

FIG. 2 presents a diagram of image-guided labeling of a living neuron using PA-GFP and DsRed in a live Drosophila brain. (A) Prior to photoactivation, the PA-GFP protein (green) is undetectable in C133 neurons (red). (B) A single neuron after two-photon laser stimulation at 820 nm (equivalent to a single photon at about 413 nm); the PA-GFP protein turns green and diffuses, filling the entire neuron. The blue flash indicates the photocativated region. This fly carries C133-Gal4, UAS-DsRed and UAS-PA-GFP.

The advantages this invention provides to scientific research are as follows. A new technology for tracing any neural circuits in the fly brain is established: PA-GFP is a photoactivatable green fluorescence protein that, after intensive irradiation with about 413-nm light, increases fluorescence by approximately 100-fold when excited by 488-nm light and remains stable for days under aerobic conditions. Transgenic flies carrying PA-GFP have been generated for image-guided tracing of neurons in the entire brain (FIG. 2). A set of brain neurons expressing PA-GFP and DsRed generates only red fluorescence until specific neurons are photoactivated by a two-photon laser to generate green fluorescence. The photoactivated PA-GFP at the cell body diffused rapidly for labeling the entire neuron without spill over to neighboring neurons above or below the focal point.

The present invention also provides a transgenic Drosophila line with genes of fluorescent proteins for exploring the directions of neural cell during development. The present invention can be applied for tracking development of a target neuron, connections with other neurons, and the establishment of neural circuits.

Research investigating the different fluorescence colors labeled on different living proteins that conform to the variations in syntheses, pathways and interactions are very interesting, it is also very time consuming. Conclusions are typically drawn only following careful experimental operations, and repeated experiments are required to overcome variability. In particular, in transgenic experiments, the overexpressed target protein and fluorescence proteins can generate abnormal distributions. For example, the DsRed fusion proteins are only present in cytoplasm, and are transferred into the nucleus when overexpressed. Additionally, protein-protein interactions can be influenced by the sizes and structures of the fluorescence proteins, and the normal functions of cells may be altered. Therefore, these factors must be addressed when designing reliable experiments.

The research investigating the development of the neuronal system in Drosophila can apply to neural systems of higher animals, such as the proneural genes atonal and amos. Homologues genes have been identified in higher animals and their functions drive the formation of neural circuits with mechanisms similar to those in driving pathway. Drosophila has a simple structure. Experimentation using Drosophila is easy and the experimental results can be extended. For example, the comparisons among different artificial neural cells driven by different proneural genes has been examined in Drosophila, and similar mechanisms are believed to occur in higher animals.

According to another view, the present invention, the present invention is not limited to Drosophila or its embryo and can also apply to other model systems, such as those applied for fish and mouse, under the spirit and scope of the present invention and the model system can be modified for experimental requirements.

For each item mentioned above, the advantages include: (A) a confocal imaging procedure that can maximize excitation efficiency without damaging living tissues; (B) a possibility to reconstruct the hard wiring within the entire brain. Although many techniques, such as dye-fills with electrodes and viral anterograde and retrograde labeling, have been developed for tracing single neurons in the brain, none are transgenic and facilitate tracing for genetically and visually identifiable neurons. Targeted two-photon irradiation and rapid intracellular diffusion of phtoactivatable PA-GFP has facilitated labeling of selected neurons in the brain and all fibers innervating any selected region of interest. Using the fly brain as a model system, the present invention may be extended to reconstruct the first complete brain neural network in any animals.

The major applications of the present invention are as follows. (1) The neural networks mapped out have anatomical significance for teaching neuroscience, and can be exercised by students. (2) The present invention provides a reliable platform for neural researchers investigating anatomy. Such a platform can be used to track the distribution, origin and target of any neuron with PA-GFP to construct detailed signal transmission pathways in the whole brain. (3) The image of neural networks can be used to evaluate drug efficacy by combining the invention with pharmacological examination protocols. The pharmaceutical industry may benefit from the knowledge of how a specific cellular morphology with specific genes is altered, damaged or repaired under medicinal treatments. At present, no methods exist that combine tracing of specific cellular structures with transgenic technologies to provide these applications. Therefore, it is reasonable to estimate that the value of these applications exceeds hundreds of millions of dollars in view of neurophamacological studies of the brain.

As will be understood by persons skilled in the art, the foregoing preferred embodiment of the present invention illustrates the present invention rather than limiting the present invention. Having described the invention in connection with a preferred embodiment, modifications will be suggested to those skilled in the art. Thus, the invention is not to be limited to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. A transgenic Drosophila line with genes of first fluorescent proteins and genes of second fluorescent proteins, wherein said genes of said first fluorescent proteins are utilized to define a predetermined area of tissues or a predetermined number of cells or neurons in said transgenic Drosophila line with a first exciting light source, and said genes of said second fluorescent proteins are utilized to identify specific cells or neurons within said predetermined area of tissues or among said predetermined number of cells or neurons with a second exciting light source.

2. The transgenic Drosophila line of claim 1, wherein said first fluorescent protein comprises red fluorescent protein.

3. The transgenic Drosophila line of claim 2, wherein said red fluorescent protein comprises DsRed.

4. The transgenic Drosophila line of claim 1, wherein said second fluorescent protein possesses differentiable optical spectrum characters to said first fluorescent protein and comprises green fluorescent protein.

5. The transgenic Drosophila line of claim 4, wherein said green fluorescent protein comprises PA-GFP.

6. The transgenic Drosophila line of claim 1, wherein the wavelength of said first exciting light source is about 558 nm or other suitable wavelengths.

7. The transgenic Drosophila line of claim 1, wherein the wavelength of said second exciting light source is about 488 nm or other suitable wavelengths.

8. The transgenic Drosophila line of claim 1, wherein said genes of second fluorescent proteins are activated by an activation light source before being excited by said second exciting light source.

9. The transgenic Drosophila line of claim 8, wherein the wave length of said activation light source is about 413 nm or other suitable wavelengths.

10. The transgenic Drosophila line of claim 8, wherein the wave length of said activation light source is about 820 nm or other suitable wavelengths.


SEQUENCE LISTING (2 sequences)

SEQ ID NO: 1 - DNA, 15 bp, Artificial Sequence
  Description of Artificial Sequence  Synthetic oligonucleotide primer
atggtgagcaagggc

SEQ ID NO: 2 - DNA, 15 bp, Artificial Sequence
  Description of Artificial Sequence  Synthetic oligonucleotide primer
ttacttgtacagctc